(19) United States
(12) Patent Application Publication
(10) Pub. No.: US 2026/0287221 A1
(43) Pub. Date: Sep. 24, 2026

(54) THERMAL CHAMBER TYPE COOLING PLATE STRUCTURE WITH IMPROVED COOLING PERFORMANCE

(71) Applicant: Shenb Co., Ltd., Seoul (KR)

(72) Inventor: Sunyoung KANG, Seoul (KR)

(21) Appl. No.: 19/278,308

(22) Filed: Jul. 23, 2025

(30) Foreign Application Priority Data
Mar. 18, 2025 (KR) 10-2025-0034782

(51) Int. Cl. F25B 21/04; A61F 7/00

(52) U.S. Cl. CPC F25B 21/04; A61F 7/007; A61F 2007/0052; A61F 2007/0056; A61F 2007/0075

(57) ABSTRACT

An embodiment relates to a thermal chamber type cooling plate structure with improved cooling performance, the thermal chamber type cooling plate structure including a chamber type cooling plate configured to come into direct contact with skin and absorb heat, a heat transfer cooling plate tightly attached and connected to the chamber type cooling plate and configured to absorb heat, which is generated from a needle cartridge accommodated in the heat transfer cooling plate, and heat, which is generated from a user's skin, and transfer the heat to a Peltier thermoelectric element, the Peltier thermoelectric element coupled to the heat transfer cooling plate and configured to absorb heat and transfer the heat to an opposite side, and a water block configured to circulate a coolant to discharge heat, which is discharged from the Peltier thermoelectric element, to the outside.

### CROSS-REFERENCE TO RELATED APPLICATIONS

This application claims the priority of Korean Patent Application No. 10-2025-0034782 filed on Mar. 18, 2025, in the Korean Intellectual Property Office, the disclosure of which is incorporated herein by reference.

### BACKGROUND

### Field

The present disclosure relates to a thermal chamber type cooling plate structure with improved cooling performance, and more particularly, to a thermal chamber type cooling plate structure with improved cooling performance, the thermal chamber type cooling plate structure being capable of effectively absorbing heat, which is generated from a needle cartridge, and heat, which is transferred from skin, dissipating heat to the outside, mitigating pain caused by heat generated during a treatment by means of a highly thermally conductive metallic material and efficient heat transfer design, and improving stability and reliability of a device.

### Description of the Related Art

In general, a handpiece of an RF needle cartridge type refers to a device that is used for skin treatment or medical procedures by mounting a needle cartridge, which transmits high frequency (RF), on a handpiece main body. The handpiece provides a therapeutic effect to skin or tissue by transferring high-frequency energy or inserting a needle precisely in a state in which the needle cartridge is fastened to the handpiece.

In this case, in the case of a skin treatment process using an RF needle cartridge in the related art, there is a problem in that a physical distance between a cooling plate and a Peltier thermoelectric element is long, which elongates a heat transfer path. For this reason, the cooling effect created by the Peltier thermoelectric element cannot be sufficiently transferred to the cooling plate that comes into contact with the skin, which causes a deterioration in cooling efficiency.

In addition, because the cooling plate cannot stably maintain a skin temperature, there is a limitation in mitigating skin damage or pain caused by heat generated during the treatment. For this reason, there is a problem in that the safety and effect of the treatment deteriorate, and the reliability of a cooling system deteriorates.

### Document of Related Art

### Patent Document

(Patent Document 1) Korean Patent Application Laid-Open No. 10-2023-0142729 (published on Oct. 11, 2023)

### SUMMARY

The present disclosure has been made in an effort to solve the above-mentioned problem, and an object of the present disclosure is to provide a thermal chamber type cooling plate structure with improved cooling performance, the thermal chamber type cooling plate structure being capable of effectively absorbing heat, which is generated from a needle cartridge, and heat, which is transferred from skin, dissipating heat to the outside, mitigating pain caused by heat generated during a treatment by means of a highly thermally conductive metallic material and efficient heat transfer design, and improving stability and reliability of a device.

The object to be achieved by the present specification is not limited to the above-described matters and may be extended to various matters that may be derived by embodiments of the present disclosure described below.

A thermal chamber type cooling plate structure 100 with improved cooling performance according to an embodiment of the present disclosure may include: a chamber type cooling plate (110) configured to come into direct contact with skin and absorb heat; a heat transfer cooling plate (120) tightly attached and connected to the chamber type cooling plate (110) and configured to absorb heat, which is generated from a needle cartridge accommodated in the heat transfer cooling plate (120), and heat, which is generated from a user's skin, and transfer the heat to a Peltier thermoelectric element (130); the Peltier thermoelectric element (130) coupled to the heat transfer cooling plate (120) and configured to absorb heat and transfer the heat to an opposite side; and a water block (140) configured to circulate a coolant to discharge heat, which is discharged from the Peltier thermoelectric element (130), to the outside, in which the chamber type cooling plate (110) and the heat transfer cooling plate (120) are made of a metallic material, and in which the Peltier thermoelectric element (130) is configured to absorb heat, which is transferred from the heat transfer cooling plate (120), and discharge the heat to the outside through the water block (140).

According to the embodiment of the present disclosure, a needle extension hole (111), through which a needle of the needle cartridge is extended to the outside, may be formed in the chamber type cooling plate (110).

According to the embodiment of the present disclosure, the chamber type cooling plate (110) may be made of an aluminum alloy or copper material, and an anodizing coating for preventing oxidation may be applied to a surface of the chamber type cooling plate (110).

According to the embodiment of the present disclosure, an inside of the heat transfer cooling plate (120) may include a flat contact surface tightly attached to the needle cartridge so that the heat transfer cooling plate (120) is configured to be in direct contact with a heat transfer portion of the needle cartridge, and a fine pattern may be processed on the contact surface to improve heat transfer efficiency.

According to the embodiment of the present disclosure, an upper surface of the Peltier thermoelectric element (130) may include a fixing slot coupled to the heat transfer cooling plate (120), a lower surface of the Peltier thermoelectric element (130) may include a mounting protruding portion coupled to the water block (140), and the Peltier thermoelectric element (130) may be configured to be detachable from the heat transfer cooling plate (120) and the water block (140).

According to the embodiment of the present disclosure, the Peltier thermoelectric element (130) may be formed in a circular ring shape with an opened central portion to correspond to a lower shape of the heat transfer cooling plate (120).

According to the embodiment of the present disclosure, the water block (140) may include a plurality of cooling fins disposed therein, and the cooling fins may be configured to absorb heat transferred from the Peltier thermoelectric element (130).

According to the embodiment of the present disclosure, the water block (140) may include a flow path design in which a coolant inlet and a coolant outlet are symmetrically disposed.

According to the embodiment of the present disclosure, a thermally conductive bonding agent may be used between the heat transfer cooling plate (120) and the Peltier thermoelectric element (130) to reduce contact resistance between the heat transfer cooling plate (120) and the Peltier thermoelectric element (130).

According to the embodiment of the present disclosure, the water block (140) may include a temperature sensor therein, and the temperature sensor may detect a coolant temperature and provide the coolant temperature to an external control system in real time.

According to the embodiment of the present disclosure, a concave-convex structure may be formed on an outer surface of the chamber type cooling plate (110) to increase a contact force with the user's skin contact surface.

According to the embodiment of the present disclosure, the heat transfer cooling plate (120) may have a multilayer structure including a thermal conduction layer and a mechanical support layer.

According to the embodiment of the present disclosure, a non-thermally conductive insulating material may be inserted between the chamber type cooling plate (110) and the heat transfer cooling plate (120) to restrict a heat transfer path.

According to the embodiment of the present disclosure, the water block (140) may include a flow rate adjustment valve in an inner flow path, and the water block (140) may be configured to adjust a flow velocity of the coolant by means of the valve.

according to another embodiment of the present disclosure may include: a chamber type cooling plate (110) configured to come into direct contact with skin and receive heat; a heat transfer cooling plate (120) tightly attached and coupled to the chamber type cooling plate (110) and configured to simultaneously absorb heat, which is generated from a needle cartridge accommodated in the heat transfer cooling plate (120), and heat generated from the skin; a Peltier thermoelectric element (130) fixedly coupled to the heat transfer cooling plate 120 and configured to transfer heat, which is absorbed by the heat transfer cooling plate (120), in an opposite direction; and a water block (140) provided to be in contact with the Peltier thermoelectric element (130) and having a cooling fin and a coolant flow path formed therein to discharge heat, which is discharged from the Peltier thermoelectric element (130), to the outside, in which the chamber type cooling plate (110) and the heat transfer cooling plate (120) are made of a metallic material with high thermal conductivity, in which a flow rate adjustment valve configured to adjust a flow of a coolant is disposed in the water block (140), and in which the Peltier thermoelectric element (130) is configured such that a thermally conductive bonding agent is interposed between a contact portion of the Peltier thermoelectric element (130) and the heat transfer cooling plate (120) to reduce contact resistance and improve heat transfer efficiency.

According to the embodiment of the present disclosure, the chamber type cooling plate and the heat transfer cooling plate are designed to be made of highly thermally conductive metallic materials and tightly attached and coupled to the Peltier thermoelectric element, thereby providing an advantage of shortening the heat transfer path and maximizing the cooling efficiency.

In addition, according to the present disclosure, with the improved cooling performance, excessive heat, which is generated during the RF needle treatment, is quickly absorbed and dissipated to the outside, thereby providing an advantage of effectively mitigating skin pain and damage.

In addition, according to the present disclosure, the thermally conductive bonding agent is used between the Peltier thermoelectric element and the heat transfer cooling plate, such that the contact resistance is reduced, and the heat transfer efficiency is improved, thereby providing an advantage of enhancing the stability and reliability of the system.

In addition, according to the present disclosure, the flow rate adjustment valve and the temperature sensor are added to the water block to finely control the flow and temperature of the coolant, thereby providing an advantage of enabling customized cooling settings in accordance with treatment environments.

In addition, according to the present disclosure, the insulating material is inserted between the chamber type cooling plate and the heat transfer cooling plate to prevent unnecessary heat transfer and maximize cooling efficiency, thereby providing an advantage of minimizing a loss of energy.

In addition, according to the present disclosure, the constituent elements are modularized and designed, such that the maintenance of the device and the replacement of the needle cartridge are facilitated, thereby providing an advantage of improving user convenience.

The effects of the present specification are not limited to the above-described matters, and it should be understood that the effects may be extended to various contents that may be derived from the following detailed descriptions of the embodiments of the present disclosure.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a view illustrating an overall shape of a thermal chamber type cooling plate structure 100 with improved cooling performance according to an embodiment of the present disclosure;

FIG. 2 is an exploded perspective view illustrating the thermal chamber type cooling plate structure 100 with improved cooling performance illustrated in FIG. 1;

FIG. 3 is a view more specifically illustrating a structure of a cooling plate 110;

FIG. 4 is a view more specifically illustrating a structure of a heat transfer cooling plate 120;

FIG. 5 is a view more specifically illustrating a structure of a Peltier thermoelectric element 130;

FIG. 6 is a view more specifically illustrating a structure of a water block 140; and

FIG. 7 is a view illustrating a temperature difference when a general cooling plate in the related art is in contact with skin and a temperature difference when the chamber type cooling plate 110 of the thermal chamber type cooling plate structure 100 with improved cooling performance according to the present disclosure is in contact with skin.

### DETAILED DESCRIPTION OF THE EMBODIMENT

Hereinafter, the exemplary embodiment of the present disclosure will be described with reference to the accompanying drawings and exemplary embodiments as follows. Scales of components illustrated in the accompanying drawings are different from the real scales for the purpose of description, so that the scales are not limited to those illustrated in the drawings.

In the description of the embodiment of the present specification, the specific descriptions of publicly-known configurations or functions will be omitted when it is determined that the specific descriptions may obscure the subject matter of the embodiment of the present specification. Further, a part irrelevant to the description of the embodiment of the present specification will be omitted in the drawings, and similar parts will be designated by similar reference numerals.

In the embodiment of the present specification, a configuration in which one constituent element is described as being “connected,” “coupled,” or “attached” to another constituent element may include not only a direct connection relationship but also an indirect connection relationship in which still another constituent element is present between the constituent elements. In addition, when any one constituent element is said to “include” or have” another constituent element, this is to be understood to mean that still another constituent element may be further provided without excluding another constituent element unless explicitly described to the contrary.

In the embodiment of the present specification, the terms such as “first” and “second” are used only to distinguish one constituent element from another constituent element and do not limit the order, importance, or the like between the elements, unless specially stated otherwise. Therefore, within the scope of the embodiment of the present specification, a first constituent element in an embodiment may be referred to as a second constituent element in another embodiment, and likewise, a second constituent element in an embodiment may be referred to as a first constituent element in another embodiment.

In the embodiment of the present specification, the constituent elements distinguished from one another are provided to clearly describe respective characteristics and not to mean that the constituent elements are necessarily separated. That is, the plurality of constituent elements may be integrated into and implemented as a single hardware or software unit, or one constituent element may be dispersed and implemented as a plurality of hardware or software units. Therefore, although not separately stated, the above-mentioned integrated or dispersed embodiment is also included in the scope of the embodiment of the present specification.

The constituent elements described in various embodiments do not necessarily mean essential constituent elements, and some of the constituent elements may be optional constituent elements. Therefore, the embodiment including the subset of the constituent elements described in the embodiment is also included in the scope of the embodiment of the present specification. In addition, the embodiment, which additionally includes other constituent elements in addition to the constituent elements described in various embodiments, is also included in the scope of the embodiment of the present specification.

Hereinafter, the embodiments of the present specification will be described in detail with reference to the drawings.

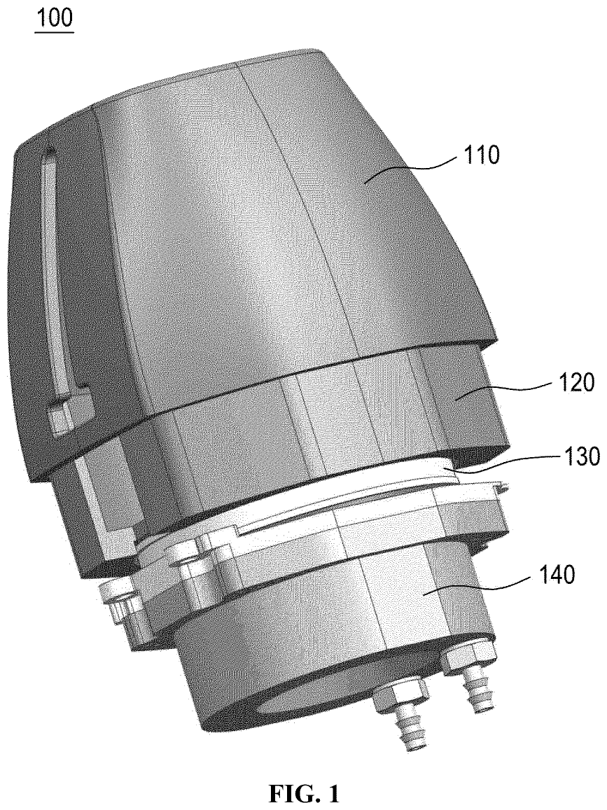

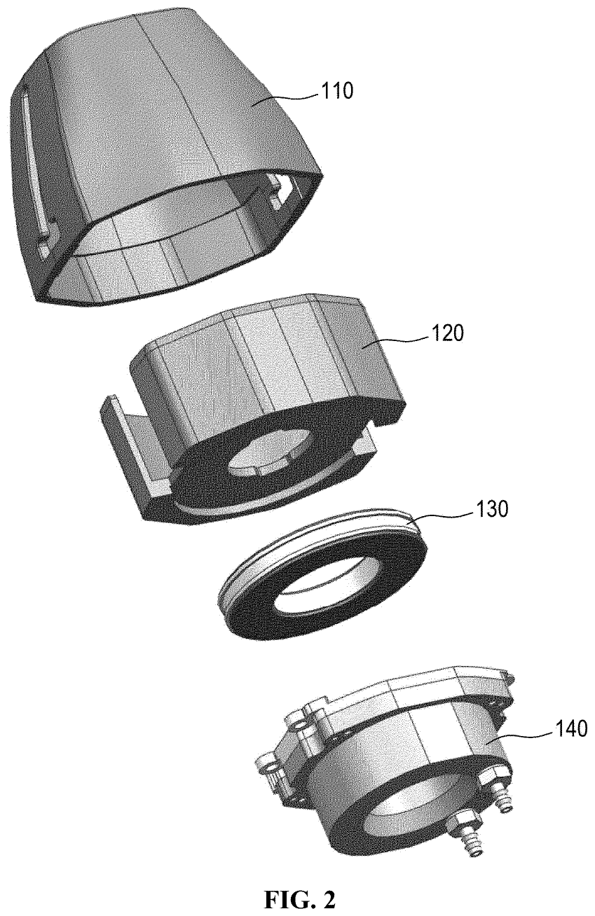

FIG. 1 is a view illustrating an overall shape of a thermal chamber type cooling plate structure 100 with improved cooling performance according to an embodiment of the present disclosure, and FIG. 2 is an exploded perspective view illustrating the thermal chamber type cooling plate structure 100 with improved cooling performance illustrated in FIG. 1.

With reference to FIGS. 1 and 2, the thermal chamber type cooling plate structure 100 with improved cooling performance according to the embodiment of the present disclosure broadly includes a chamber type cooling plate 110, a heat transfer cooling plate 120, a Peltier thermoelectric element 130, and a water block 140.

The chamber type cooling plate 110 may serve to absorb heat while coming into direct contact with skin. This configuration will be described more specifically.

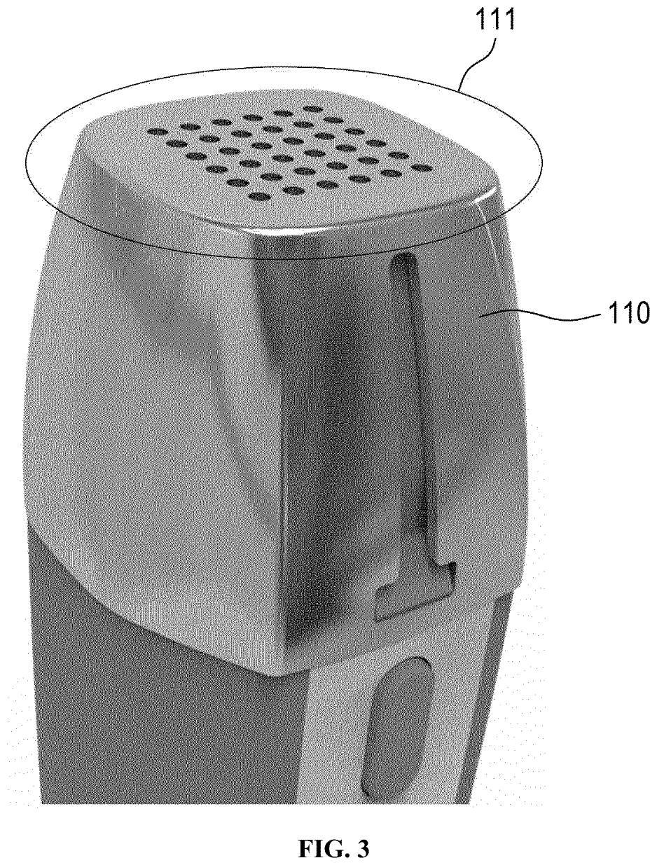

FIG. 3 is a view more specifically illustrating a structure of the cooling plate 110.

With reference to FIG. 3, the chamber type cooling plate 110 is positioned at an upper end of an RF needle treatment device and serves to efficiently absorb heat, which is generated while a needle cartridge and the skin are in contact with each other, and transfer heat to the heat transfer cooling plate 120 to be described below. The plate may be made of a metallic material (e.g., aluminum alloy, copper, etc.) and has high thermal conductivity. An anodizing coating may be additionally applied to a surface of the plate to enhance durability and prevent oxidation.

In addition, a plurality of needle extension holes 111 are formed at an upper end of the chamber type cooling plate 110 so that needles of the needle cartridge protrude toward the skin. The needle extension holes 111 are precisely arranged and processed so that the needles may smoothly pass through the needle extension holes 111. A hole peripheral portion is smoothly finished to maintain heat transfer efficiency.

In addition, the inside of the chamber type cooling plate 110 includes a flat surface contact portion having a uniform thickness to optimize heat transfer and precisely processed so that the flat surface contact portion may be maximally tightly attached and coupled to the needle cartridge and the heat transfer cooling plate 120 to be described below.

In addition, an outer peripheral shape of the chamber type cooling plate 110 may be a shape that may be tightly coupled to a handpiece main body. An edge of an uppermost end surface of the chamber type cooling plate 110, which comes into contact with the skin, is curved to be slightly rounded, such that the chamber type cooling plate 110 may be softly and tightly attached to the user's skin.

In this case, an overall external appearance of the above-mentioned chamber type cooling plate 110 may be a dome-shaped chamber. More particularly, the dome shape may be configured such that four peripheral side surfaces may define an isosceles trapezoid, and an upper end of the dome shape may have a shape cut flat.

The chamber type cooling plate 110 may come into contact with the skin and serve to absorb heat, which is generated from the needle cartridge and the skin, and efficiently transfer the heat to the heat transfer cooling plate 120. The chamber type cooling plate 110 may serve to provide a cooling effect while coming into direct contact with the skin, thereby preventing skin damage caused by excessive heat, which is generated during the treatment, and mitigating pain.

The heat transfer cooling plate 120 is tightly attached and connected to the chamber type cooling plate 110 and serves to absorb heat, which is generated from the needle cartridge accommodated in the heat transfer cooling plate 120, and heat, which is generated from the user's skin, and transfer the heat to the Peltier thermoelectric element 130. This configuration will be described more specifically.

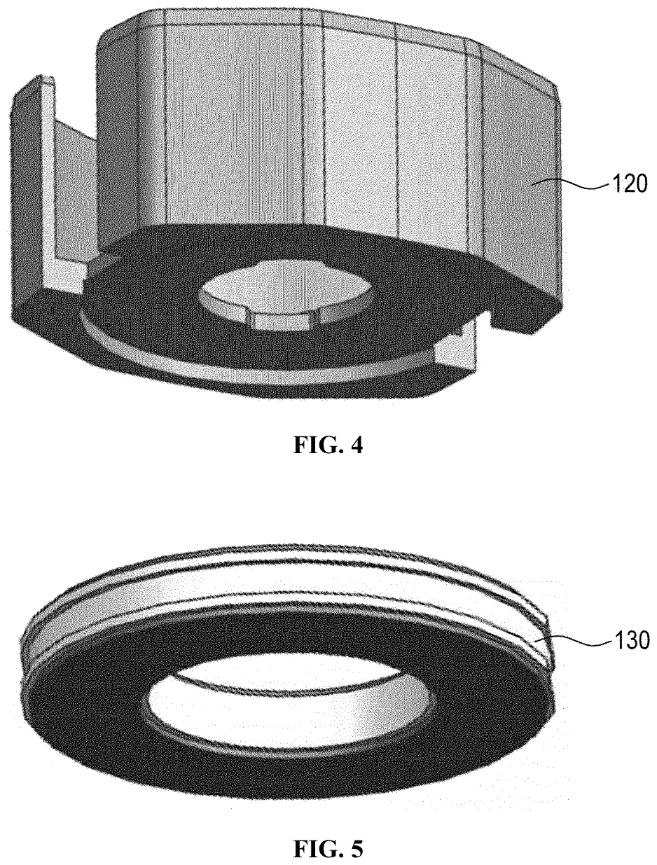

FIG. 4 is a view more specifically illustrating a structure of the heat transfer cooling plate 120.

With reference to FIG. 4, in order to efficiently transfer heat generated during the RF needle treatment, the heat transfer cooling plate 120 is tightly attached and coupled to the chamber type cooling plate 110 and serves to absorb heat, which is generated from the needle cartridge and the skin, and transfer the heat to the Peltier thermoelectric element 130 to be described below. The heat transfer cooling plate 120 may be made of a highly thermally conductive metallic material to maximize heat transfer efficiency and improve performance in cooling the treatment device by means of precise processing and design.

A flat contact surface may be formed at an upper end of the heat transfer cooling plate 120 so that the needle cartridge accommodated in the heat transfer cooling plate 120 is directly and tightly attached to the flat contact surface. The contact surface may be precisely processed to improve heat transfer efficiency. A fine pattern or a groove may be additionally processed to increase a surface area, such that the heat generated from the needle cartridge may be maximally absorbed and transferred to the Peltier thermoelectric element 130.

In addition, a lower end of the heat transfer cooling plate 120 is structured to be tightly attached and coupled to the Peltier thermoelectric element 130 to be described below and has a shape consistent with an upper surface of the Peltier thermoelectric element 130. In addition, a thermally conductive bonding agent or thermal pad may also be applied to a lower end contact surface of the heat transfer cooling plate 120 to improve efficiency in heat transfer with the Peltier thermoelectric element 130.

The heat transfer cooling plate 120 may be made of a highly thermally conductive material such as an aluminum alloy or copper, and an anodizing coating or a heat dissipation coating may be additionally applied to a surface of the heat transfer cooling plate 120 to prevent oxidation.

In addition, the heat transfer cooling plate 120 may be tightly attached and coupled to the chamber type cooling plate 110. The heat transfer cooling plate 120 and the chamber type cooling plate 110 may be coupled in a slide-insertion manner. The heat transfer cooling plate 120 and the chamber type cooling plate 110 may be fastened to each other by caught and coupled at one side. Therefore, a heat transfer path may be minimized, and the thermal conduction efficiency may be maximized. In addition, the heat transfer cooling plate 120 may be designed to be fixed in the handpiece main body and stably coupled to the main body by means of a fixing screw or a clamp structure.

In addition, in the embodiment, the heat transfer cooling plate 120 may have a multilayer structure including a thermal conduction layer and a mechanical support layer to enhance heat transfer performance, as necessary. Therefore, it is possible to improve the durability of the heat transfer cooling plate 120 while maintaining the heat transfer performance. An insulating material may be additionally inserted between the heat transfer cooling plate 120 and the chamber type cooling plate 110 or the Peltier thermoelectric element 130 to prevent an unnecessary leak of heat.

The Peltier thermoelectric element 130 is coupled to the heat transfer cooling plate 120 and serves to absorb heat and transfer the heat to the opposite side. This configuration will be described more specifically.

FIG. 5 is a view more specifically illustrating a structure of the Peltier thermoelectric element 130.

With reference to FIG. 5, the Peltier thermoelectric element 130 is positioned between the heat transfer cooling plate 120 and the water block 140 and serves to control heat transfer and dissipation. The Peltier thermoelectric element 130 operates on the basis of a principle in which heat is absorbed from one surface and discharged to the opposite side by using electrical energy.

More specifically, an upper end surface of the Peltier thermoelectric element 130 is configured to be tightly attached and coupled to the heat transfer cooling plate 120. The upper end surface of the Peltier thermoelectric element 130 may be formed as a flat contact surface to absorb heat and precisely processed to maximize thermal conduction efficiency.

In addition, a lower end surface of the Peltier thermoelectric element 130 may be configured to be coupled to the water block 140 to be described below and processed as a flat shape that may be subjected to stable contact to dissipate heat. In addition, a thermally conductive bonding agent or thermal pad may be disposed between the Peltier thermoelectric element 130 and the water block 140 to minimize contact resistance between the lower end surface of the Peltier thermoelectric element 130 and the water block 140.

In addition, the Peltier thermoelectric element 130 may include a plurality of thermoelectric materials (semiconductors) therein, and an upper end and a lower end of the Peltier thermoelectric element 130 may be configured to electrically and independently operate. With this configuration, the Peltier thermoelectric element 130 may simultaneously perform heat absorption and heat dissipation. In addition, the Peltier thermoelectric element 130 may be manufactured by using a semiconductor material and an electrode made of a highly thermally conductive material, and a size and thickness of the Peltier thermoelectric element 130 may be optimized in consideration of heat transfer efficiency and durability.

The Peltier thermoelectric element 130 may absorb heat transferred from the heat transfer cooling plate 120 through an upper end surface thereof, and the absorbed heat may be discharged to the water block 140 through the lower end surface and transferred to the outside by means of circulation of a coolant in the water block. In addition, the Peltier thermoelectric element 130 may mitigate pain and prevent skin damage during the treatment by lowering temperatures of the cooling plate and the skin by efficiently transferring heat by using electrical energy.

In addition, in the embodiment, the Peltier thermoelectric element 130 may be made of a plurality of semiconductor materials, such that heat absorption and dissipation efficiency may be maximized. As necessary, a plurality of Peltier thermoelectric elements 130 may be disposed in parallel or in series.

In addition, to protect the Peltier thermoelectric element 130 from an electrical overload and physical impact, a protective coating or a design for improving durability may be additionally provided at an outer periphery of the Peltier thermoelectric element. Thermal conduction layers may be additionally disposed at upper and lower ends of the Peltier thermoelectric element 130, thereby assisting a thermal flow and further improving thermal efficiency.

In addition, an upper surface of the Peltier thermoelectric element 130 may include a fixing slot coupled to the heat transfer cooling plate 120, a lower surface of the Peltier thermoelectric element 130 may include a mounting protruding portion coupled to the water block 140, and the Peltier thermoelectric element 130 may be configured to be detached from the heat transfer cooling plate 120 and the water block 140.

In addition, the Peltier thermoelectric element 130 may be formed in a circular ring shape with an opened central portion to correspond to a lower shape of the heat transfer cooling plate 120.

The water block 140 is configured to circulate the coolant to discharge heat, which is discharged from the Peltier thermoelectric element 130, to the outside and efficiently discharged to heat, which is discharged from the Peltier thermoelectric element 130, to provide a consistent cooling effect. This configuration will be described more specifically.

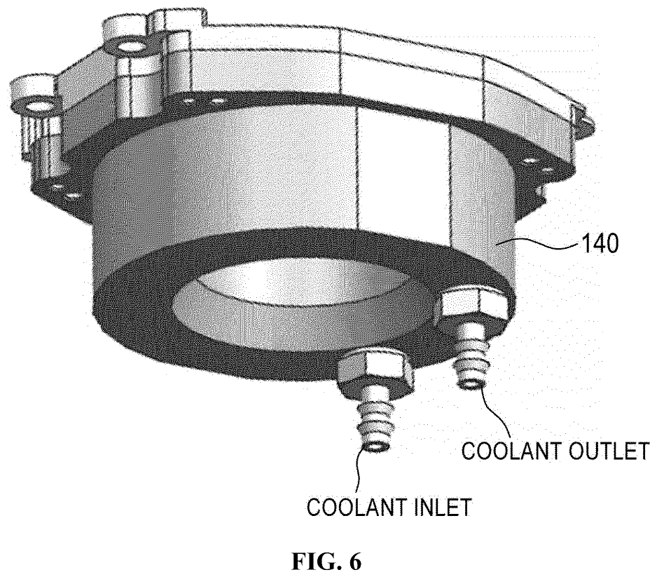

FIG. 6 is a view more specifically illustrating a structure of the water block 140.

With reference to FIG. 6, the water block 140 serves to dissipate heat, which is transferred from the Peltier thermoelectric element 130, to the outside by circulating the coolant. The water block 140 is tightly attached and coupled to the Peltier thermoelectric element 130 and serves to efficiently adjust a skin temperature during the RF needle treatment by effectively controlling the heat dissipation process.

A plurality of cooling fins may be disposed in the water block 140. The cooling fin serves to absorb heat transferred from the Peltier thermoelectric element 130 and improve heat transfer efficiency by maximizing a contact area with the coolant. In addition, the cooling fin may be made of highly thermally conductive metal (e.g., copper, aluminum). In the embodiment, the cooling fin may be designed to have a rib or concave-convex structure to increase the surface area.

In addition, a flow path (passageway) may be designed in the water block 140 to circulate the coolant. The flow path is configured to perform heat exchange while passing between the cooling fins. The flow path is configured to minimize flow resistance of the coolant.

In addition, a coolant inlet and a coolant outlet may be symmetrically disposed on an outer portion of the water block 140. In this case, the inlet and the outlet may be coupled to a connection part (e.g., a screw type connector) so that the coolant may dissipate heat to the outside while circulating.

In addition, the outer portion of the water block 140 may be made of a secure metallic material to enhance durability, and a rustproof coating or heat-resistant coating may be applied to a surface of the outer portion of the water block 140 to ensure corrosion resistance. In addition, a flat contact surface may be formed on the outer portion of the water block 140 so that the water block 140 is coupled to the Peltier thermoelectric element 130.

In addition, a temperature sensor may be additionally mounted inside or outside the water block 140. The temperature sensor may monitor a temperature of the coolant in real time and provide the temperature of the coolant to an external control system. In addition, the water block 140 may include a flow rate adjustment valve configured to control a flow velocity of the coolant. The flow rate adjustment valve optimizes the cooling performance by finely adjusting the flow of the coolant.

The water block 140 may dissipate heat, which is transferred from the Peltier thermoelectric element 130, to the outside by exchanging heat with the coolant, thereby maintaining thermal balance and preventing the skin from being overheated during the RF needle treatment. In addition, the water block 140 may allow the coolant to absorb heat efficiently and circulate by means of the internal cooling fins and the flow path structure, thereby maximizing cooling efficiency.

Next, a result of comparing temperature differences when the cooling plates come into contact with the skin by using a general cooling plate in the related art and the chamber type cooling plate 110 of the thermal chamber type cooling plate structure 100 with improved cooling performance according to the present disclosure will be described.

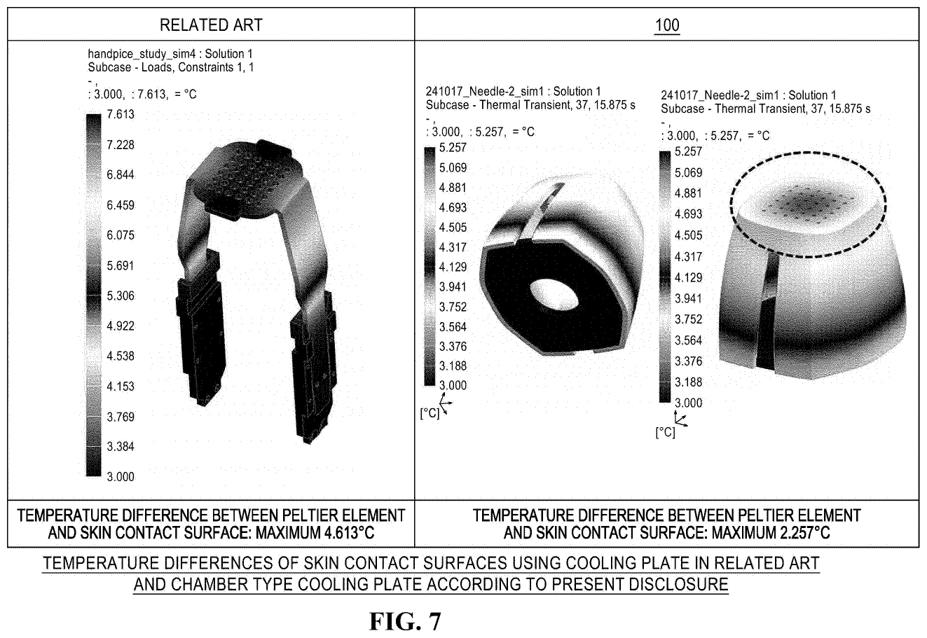

FIG. 7 is a view illustrating a temperature difference when the general cooling plate in the related art is in contact with skin and a temperature difference when the chamber type cooling plate 110 of the thermal chamber type cooling plate structure 100 with improved cooling performance according to the present disclosure is in contact with skin.

First, with reference to FIG. 7A, it can be seen that in the cooling plate structure in the related art, a temperature difference between the Peltier thermoelectric element and the skin contact surface is up to 4.613° C., and a local high-temperature occurs in which heat is concentrated in a particular region. This is based on the fact that heat transfer efficiency is low and heat absorbed by the Peltier thermoelectric element cannot be effectively dispersed to the skin contact surface. As a result, this is likely to cause skin irritation and damage.

In contrast, with reference to FIG. 7B, it can be seen that the thermal chamber type cooling plate structure 100 according to the present disclosure significantly improves cooling performance by reducing a temperature difference between the Peltier thermoelectric element and the skin contact surface according to at most 2.257° C. In particular, it can be seen that with the metallic materials and structural optimization of the chamber type cooling plate 110 and the heat transfer cooling plate 120, heat may be uniformly distributed to the entire skin contact surface, and the local high-temperature phenomenon may be effectively mitigated.

With the above-mentioned improvement, the thermal chamber type cooling plate structure 100 with improved cooling performance according to the present disclosure may reduce the temperature difference by about 51% (4.613° C. to 2.257° C.) in comparison with the related art, which may not only improve the cooling performance but also significantly mitigate the user's pain and improve safety by stably maintaining the temperature of the skin contact surface during the RF needle treatment.

While the present disclosure has been described in connection with some embodiments in the present specification, various modifications and alterations may be made without departing from the scope of the present disclosure as understood by those skilled in the art to which the present disclosure pertains. In addition, the modifications and alterations need to be considered as falling within the scope of the claims attached to the present specification.

### Description of Reference Numerals

100: Thermal chamber type cooling plate structure with improved cooling performance
110: Chamber type cooling plate
120: Heat transfer cooling plate
130: Peltier thermoelectric element
140: Water block

What is claimed is:

1. A thermal chamber type cooling plate structure with improved cooling performance, the thermal chamber type cooling plate structure comprising:
a chamber type cooling plate (110) configured to come into direct contact with skin and absorb heat;
a heat transfer cooling plate (120) tightly attached and connected to the chamber type cooling plate (110) and configured to absorb heat, which is generated from a needle cartridge accommodated in the heat transfer cooling plate (120), and heat, which is generated from a user's skin, and transfer the heat to a Peltier thermoelectric element (130);
the Peltier thermoelectric element (130) coupled to the heat transfer cooling plate (120) and configured to absorb heat and transfer the heat to an opposite side; and
a water block (140) configured to circulate a coolant to discharge heat, which is discharged from the Peltier thermoelectric element (130), to the outside,
wherein the chamber type cooling plate (110) and the heat transfer cooling plate (120) are made of a metallic material, and
wherein the Peltier thermoelectric element (130) is configured to absorb heat, which is transferred from the heat transfer cooling plate (120), and discharge the heat to the outside through the water block (140).

2. The thermal chamber type cooling plate structure of claim 1, wherein a needle extension hole (111), through which a needle of the needle cartridge is extended to the outside, is formed in the chamber type cooling plate (110).

3. The thermal chamber type cooling plate structure of claim 2, wherein the chamber type cooling plate (110) is made of an aluminum alloy or copper material, and an anodizing coating for preventing oxidation is applied to a surface of the chamber type cooling plate (110).

4. The thermal chamber type cooling plate structure of claim 3, wherein an inside of the heat transfer cooling plate (120) comprises a flat contact surface tightly attached to the needle cartridge so that the heat transfer cooling plate (120) is configured to be in direct contact with a heat transfer portion of the needle cartridge, and
wherein a fine pattern is processed on the contact surface to improve heat transfer efficiency.

5. The thermal chamber type cooling plate structure of claim 4, wherein an upper surface of the Peltier thermoelectric element (130) comprises a fixing slot coupled to the heat transfer cooling plate (120), a lower surface of the Peltier thermoelectric element (130) comprises a mounting protruding portion coupled to the water block (140), and the Peltier thermoelectric element (130) is configured to be detachable from the heat transfer cooling plate (120) and the water block (140).

6. The thermal chamber type cooling plate structure of claim 5, wherein the Peltier thermoelectric element (130) is formed in a circular ring shape with an opened central portion to correspond to a lower shape of the heat transfer cooling plate (120).

7. The thermal chamber type cooling plate structure of claim 6, wherein the water block (140) comprises a plurality of cooling fins disposed therein, and the cooling fins are configured to absorb heat transferred from the Peltier thermoelectric element (130).

8. The thermal chamber type cooling plate structure of claim 7, wherein the water block (140) comprises a flow path design in which a coolant inlet and a coolant outlet are symmetrically disposed.

9. The thermal chamber type cooling plate structure of claim 8, wherein a thermally conductive bonding agent is used between the heat transfer cooling plate (120) and the Peltier thermoelectric element (130) to reduce contact resistance between the heat transfer cooling plate (120) and the Peltier thermoelectric element (130).

10. The thermal chamber type cooling plate structure of claim 9, wherein the water block (140) comprises a temperature sensor therein, and the temperature sensor detects a coolant temperature and provides the coolant temperature to an external control system in real time.

11. The thermal chamber type cooling plate structure of claim 10, wherein a concave-convex structure is formed on an outer surface of the chamber type cooling plate (110) to increase a contact force with the user's skin contact surface.

12. The thermal chamber type cooling plate structure of claim 11, wherein the heat transfer cooling plate (120) has a multilayer structure comprising a thermal conduction layer and a mechanical support layer.

13. The thermal chamber type cooling plate structure of claim 12, wherein a non-thermally conductive insulating material is inserted between the chamber type cooling plate (110) and the heat transfer cooling plate (120) to restrict a heat transfer path.

14. The thermal chamber type cooling plate structure of claim 13, wherein the water block (140) comprises a flow rate adjustment valve in an inner flow path, and the water block (140) is configured to adjust a flow velocity of the coolant by means of the valve.

15. A thermal chamber type cooling plate structure with improved cooling performance, the thermal chamber type cooling plate structure comprising:
a chamber type cooling plate (110) configured to come into direct contact with skin and receive heat;
a heat transfer cooling plate (120) tightly attached and coupled to the chamber type cooling plate (110) and configured to simultaneously absorb heat, which is generated from a needle cartridge accommodated in the heat transfer cooling plate (120), and heat generated from the skin;
a Peltier thermoelectric element (130) fixedly coupled to the heat transfer cooling plate 120 and configured to transfer heat, which is absorbed by the heat transfer cooling plate (120), in an opposite direction; and
a water block (140) provided to be in contact with the Peltier thermoelectric element (130) and having a cooling fin and a coolant flow path formed therein to discharge heat, which is discharged from the Peltier thermoelectric element (130), to the outside,
wherein the chamber type cooling plate (110) and the heat transfer cooling plate (120) are made of a metallic material with high thermal conductivity,
wherein a flow rate adjustment valve configured to adjust a flow of a coolant is disposed in the water block (140), and
wherein the Peltier thermoelectric element (130) is configured such that a thermally conductive bonding agent is interposed between a contact portion of the Peltier thermoelectric element (130) and the heat transfer cooling plate (120) to reduce contact resistance and improve heat transfer efficiency.
